(19) United States
(12) Patent Application Publication
(10) Pub. No.: US 2023/0103452 A1
(43) Pub. Date: Apr. 6, 2023

(54) SYSTEMS AND METHODS FOR 4D PRINTING FOR MEMBRANOUS TISSUE FABRICATION

(71) Applicant: University of Maryland, College Park, College Park, MD (US)

(72) Inventors: John Patrick Fisher, Kensington, MD (US); Shannon Theresa McLoughlin, Silver Spring, MD (US)

(21) Appl. No.: 17/938,487

(22) Filed: Oct. 6, 2022

Related U.S. Application Data
(60) Provisional application No. 63/262,183, filed on Oct. 6, 2021.
(60) Provisional application No. 63/363,483, filed on Apr. 22, 2022.

(51) Int. Cl. A61L 27/50; A61L 27/22

(52) U.S. Cl. CPC A61L 27/50; A61L 27/222; A61L 27/52

(57) ABSTRACT

A system and method for tissue fabrication involves the use of charge manipulation between two biomaterials to generate a shrinking response, which effectively enhances the resolution of bioprinted hydrogels. The charge manipulation can be utilized to generate tissue engineered thin, membranous tissues, such as the periosteum, which is approximately one hundred microns in thickness. Thin membranous tissues in the body also have relatively complex anatomies containing multiple cell populations, and no prior strategies allow for the effective and biomimetic generation of these tissues, which can have significant impact on tissue regeneration.

### CROSS REFERENCE TO RELATED APPLICATIONS

This application claims priority under 35 U.S.C. § 119 to provisional patent applications U.S. Ser. Nos. 63/262,183, filed Oct. 6, 2021, and 63/363,483, filed Apr. 22, 2022. The provisional patent applications are herein incorporated by reference in their entireties, including without limitation, the specification, claims, and abstract, as well as any figures, tables, appendices, or drawings thereof.

### STATEMENT REGARDING FEDERALLY SPONSORED RESEARCH OR DEVELOPMENT

This invention was made with government support under P41EB023833 awarded by the National Institutes of Health (NIH). The government has certain rights in the invention.

### TECHNICAL FIELD

The presently disclosed subject matter relates generally to a system and method for tissue fabrication.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Several fabrication methods exist for tissue engineered membranes, such as casting, spin coating, electrospinning, and cell sheet engineering. Yet none of these strategies allow for the generation of multi-layered thin membranous structures with stratified cell populations.

Three dimensional (3D) extrusion bioprinting strategies have several advantages for use in tissue engineering strategies. They allow for the fabrication of complex, anatomical structures; can incorporate a broad range of materials, including both soft and hard tissue materials; and have relatively fast fabrication times. Yet, a major disadvantage of extrusion bioprinting strategies are their modest resolutions (˜150 μm). Additional disadvantages include a limited viscosity range within the materials a high shear stress to incorporate cells; and a required customization for each material type.

Other printing techniques, such as stereolithography and inkjet strategies, can achieve resolutions within the 20-50 μm range, Stereolithography and inkjet strategies allow for the generation of more biomimetic structures however are limited to single materials or the need for support materials.

The use of small diameter needles in extrusion bioprinting also results in elevated printing pressures, which can cause increased shear stress to cells incorporated within printed bio-inks, leading to greater cell death.

One particular subset of tissue structures that lack biomimetic fabrication strategies are thin membranous tissues, such as the periosteum, cornea, and epidermis. These tissues range from thicknesses of 100-600 μm and contain multiple stratified layers of different cell populations. It is because of this complex microscopic architecture that these tissues are particularly difficult to recapitulate for tissue engineering purposes. Previous strategies have investigated the use of cell sheet engineering to generate these structures, however, these strategies require significant handling of delicate cellular materials. Other strategies for generation of periosteum specifically have investigated the incorporation of multiple cell populations into a single tissue engineered structure, but these do not replicate the natural anatomical structure.

Therefore, there exists a need in the art to enhance extrusion bioprinting resolution so as to aid in the generation of biomimetic tissue structures. More particularly, there exists a need in the art for a post-fabrication processing strategy that harnesses charge interactions of anionic gelatin methacrylate (GelMA) hydrogels in poly-lysine or a cationic chitosan solution to produce a shrinking behavior.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to manipulate charge interactions between polyanionic and polycationic hydrogels to induce a shrinking behavior, thereby overcoming the issue of limited resolution associated with extrusion bioprinting. By incubating anionic hydrogels in cationic polymer solutions, charge attraction between the two polymers occurs, resulting in slight water expulsion from the matrix, and subsequent shrinking behavior

It is still yet a further object, feature, and/or advantage of the present disclosure to place printed polyanionic gelatin methacrylate (GelMA) hydrogels in polycationic chitosan hydrogel solutions. While this results in a non-permanent shape change behavior, a secondary crosslinking mechanism can be incorporated that results in shape maintenance post shrink. For example, this can be accomplished by incubating shrunken samples microbial transglutaminase (MTGase) after chitosan incubation.

It is still yet a further object, feature, and/or advantage of the present disclosure to determine the impact of the shrinking process on printed structures and to investigate whether this shape change behavior is isotropic. An isotropic change behavior lends to proportional shrinking in both printed fiber height and diameter.

It is still yet a further object, feature, and/or advantage of the present disclosure to cast samples of different thicknesses and measuring their dimensions via microscopy at each point.

It is still yet a further object, feature, and/or advantage of the present disclosure to enhance bioprinting resolution by incorporating secondary crosslinking for permanent shape change behavior.

It is still yet a further object, feature, and/or advantage of the present disclosure to use 4D printing mechanisms, such as shrinking shape change behavior, for the generation of thin membranous tissues. Typical 4D printing strategies have limited impact on biomedical applications. The methods described herein however specifically targets a subset of tissues for which there is no viable solution.

It is still yet a further object, feature, and/or advantage of the present disclosure to evaluate different anionic and cationic hydrogel combinations and investigate potential resolution enhancement for extrusion printing of hydrogels. Previous work demonstrated that cationic chitosan effectively shrinks gelatin methacrylate (GelMA) and methacrylate hyaluronic acid (HAMA) hydrogels. While chitosan acts as an efficient shrinking agent, for GelMA-based hydrogels, the acidic nature of chitosan negatively impacts cell viability. Other polycationic polymers, such as polylysine, can replace chitosan and potentially improve upon the cytocompatibility of this strategy when incorporating cells into the bio-ink.

The methods and frameworks disclosed herein can be used in a wide variety of applications. For example,

The methods for 3D bioprinting and post-processing strategies employed herein preferably produce biocompatible components in a cost-effective and reproducible manner.

3D bioprinters that print three dimensional constructs using the aforementioned methods and strategies will meet at least some, if not all, of the previously stated objectives.

According to some aspects of the present disclosure, a method of generating a shrinking response comprises allowing for cells to be incorporated into shape-changing gels; manipulating charge interactions between the shape-changing gels to induce a shrinking behavior and to expel water from the hydrogels; and generating a three-dimensional construct using an extrusion-based bioprinting process.

According to some additional aspects of the present disclosure, the method can further comprise incubating shrunken samples with a secondary crosslinking mechanism to provide a permanent shape to said shape-changing gels.

According to some additional aspects of the present disclosure, the primary crosslinking mechanism can be initiated by UV exposure. The primary crosslinking mechanism can comprise Lithium phenyl-2,4,6-trimethylbenzoylphosphinate (LAP). LAP initiates free radical polymerization upon exposure to UV light. LAP and UV light work together as the primary crosslinking mechanism.

According to some additional aspects of the present disclosure, the secondary crosslinking mechanism is an enzymatic crosslinking mechanism. The enzymatic crosslinking mechanism can comprise microbial transglutaminase (MTGase). MTGase can be used as a secondary crosslinker to maintain the shrunken state.

According to some additional aspects of the present disclosure, the shape-changing gels comprise a polyanionic hydrogel and a polycationic hydrogel. For example, the polyanionic hydrogel can comprise gelatin methacrylate (GelMA), and the polycationic hydrogel can comprise chitosan or polylysine.

According to some additional aspects of the present disclosure, the three-dimensional construct is a membranous tissue selected from the group consisting of: periosteum, cornea, and epidermis. The resolution of bioprinted hydrogels can be enhanced in the extrusion-based printing such that the membranous tissue is less than one hundred microns (100 μm) in thickness.

According to some additional aspects of the present disclosure, the three-dimensional construct comprises a bio-ink.

According to some other aspects of the present disclosure, a three-dimensional construct comprises a polyanionic hydrogel and a polycationic hydrogel; cells that can be incorporated into the polyanionic hydrogel; and a permanent shape formed from primary UV crosslinking and secondary enzymatic crosslinking.

According to some additional aspects of the present disclosure, the polyanionic hydrogel is anionic gelatin methacrylate (GelMA) and the polycationic hydrogel cationic comprises poly-L-lysine (PLL).

According to some additional aspects of the present disclosure, longer chain PLL molecules are employed to better contract GelMA hydrogel networks.

According to some other aspects of the present disclosure, a method of demonstrating a shrinking response in a three-dimensional bioprinted material comprises: synthesizing gelatin methacrylate (GelMA) from type B gelatin and methacrylic anhydride, dialyzed, and lyophilized; dissolving the GelMA in a solution with a photoinitiator; casting samples; after casting said samples, UV crosslinking the samples by activating the photoinitiator; immersed said crosslinked samples in a cationic chitosan solutions, gelatin type A, or poly-lysine.

According to some additional aspects of the present disclosure, the solution comprises gelatin methacrylate dissolved in phosphate buffered saline (PBS) at a concentration of 5% w/v with lithium phenyl-2,4,6-trimethylbenzoylphosphinate (LAP) at a concentration of 0.1% w/v.

According to some additional aspects of the present disclosure, the samples are cast in a cylindrical shape with a diameter equal to or less than 4.5 mm and a thickness equal to or less than 1.50 mm.

According to some additional aspects of the present disclosure, the method can further comprise printing at a temperature of approximately 10° C., speed of approximately 4-5 mm/s, and a pressure of approximately 6-8 psi in the shape of individual fibers.

According to some additional aspects of the present disclosure, the immersing occurs from 1-4 hours of incubation time.

In certain aspects, the present disclosure involves the use of charge manipulation between two biomaterials to generate a shrinking response, which should effectively enhance the resolution of bioprinted hydrogels. Specifically, in certain aspects, this strategy can be utilized to generate tissue engineered thin, membranous tissues, such as the periosteum, which is approximately one hundred microns (˜100 μm) in thickness. Thin membranous tissues in the body also have relatively complex anatomies containing multiple cell populations. The methods and post-processing strategies discussed herein allow for the effective and biomimetic generation of these tissues, which can have significant impact on tissue regeneration. For example, the effects can be widely applied to other types of membranous tissues, including: the tympanic membrane (3 layers that are each one hundred microns (˜100 μm)) and the epidermis (5 layers between approximately seventy (˜70 μm) and two hundred eighty microns (˜280 μm).

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 is a schematic representing a shrinking and secondary crosslinking system.

FIG. 2 is a schematic representing GelMA synthesis, primary UV crosslinking, and secondary enzymatic crosslinking.

FIG. 3 illustrates a workflow of printing/shrinking process.

FIG. 4 charts casted sample data demonstrating volume reduction from shrinking and secondary crosslinking, and more particularly: calculated volumes of casted GelMA hydrogels post cast, post shrink in chitosan solution, and post incubation in MTGase.

FIG. 5 charts casted sample data demonstrating impact of secondary crosslinking on overall volume reduction, and more particularly: percent overall volume reduction of casted gels incubated in PBS and MTGase for 24 hours post shrinking, according to certain aspects of the present disclosure. Different letters indicate statistical significance (p<0.05). Values are reported mean±standard deviation.

FIG. 6 charts casted sample data demonstrating stability of secondary crosslinking. More particularly, data of sample diameter after shrinking, secondary crosslinking in MTGase for 24 hours, and subsequent incubation in PBS for 24 hours is presented.

FIG. 7 charts casted sample data demonstrating isotropic nature of shrinking process. More particularly, sample thickness post shrink of two different hydrogel thicknesses is quantified.

FIG. 8 captures images of printed GelMA structures post print post shrink for fiber diameter measurement.

FIG. 9 charts quantification of printed fiber diameter post print and post shrink.

FIG. 10 is a schematic of osmotic pressure and charge driven shrinking mechanisms. In osmotic pressure driven shrinking, higher osmotic pressure outside the GelMA network results in water expulsion from the matrix. In charge driven shrinking, cationic PLL infiltrates GelMA network, charges attract, and PLL contracts GelMA network.

FIG. 11 shows osmotic pressure-driven shrinking only modestly shrinks GelMA network. Sample size: n=5 with 8 technical replicates; ***indicates significance of p<0.005; Mean±standard deviation.

FIG. 12 shows Poly-L-lysine contracts GelMA network. Sample size: n=5 with 8 technical replicates; ***indicates significance of p<0.005; Mean±standard deviation.

FIG. 13 demonstrates longer Poly-L-lysine molecules lead to better network collapse, regardless of number of charges or molecules in solution. Sample size: n=5 with 8 technical replicates; *indicates significance of p<0.05; ***indicates significance of p<0.005; Mean±standard deviation. (n=5, *p<0.05, **p<0.01, ***p<0.001, ****p<0.0001).

FIG. 14 captures images of Poly-L-lysine MW driving the final printed layer thickness. Post print and post shrink images of printed GelMA layers treated for 2 hours with 1 mM L-lysine, 84 kDa and 167 kDa PLL. FIG. 14 also quantifies (i) layer thickness post shrink with varied molecular weight, concentration, and time treatments with PLL. Dotted line represents needle diameter; and (ii) percent reduction after various shrinking treatments. (n=5, *p<0.05, **p<0.01, ***p<0.001).

FIG. 15 demonstrates Poly-L-lysine incubation enhances extrusion print resolution. Sample size: n=5 with 10 technical replicates; **indicates significance of p<0.01; ***indicates significance of p<0.005; Mean±standard deviation.

FIG. 16 captures extrusion-based printing can generate macroscale and microscale structures in the same construct.

FIG. 17 demonstrates longer Poly-L-lysine chains allow for increased GelMA network collapse.

FIG. 18 calculates mesh size of GelMA hydrogels treated with 2 hour shrinking using 1 mM L-lysine, 74 kDa, and 167 kDa PLL. Mesh size analysis was conducted using the Flory-Rehner theory for hydrogels crosslinked in the presence of water. FIG. 18 also shows a schematic depicting effect of PLL molecular weight on mesh size. (n=5, ****p<0.0001).

FIG. 19 shows a representative tissue engineering periosteum print, according to some aspects of the present disclosure.

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

Extrusion-based bioprinting (EBB) allows for the fabrication of constructs for bone tissue engineering (BTE). With this technique, cell-laden hydrogels or bio-inks have been extruded onto printing stages, layer-by-layer, to form three-dimensional (3D) constructs with varying sizes, shapes, and resolutions.

Extrusion bioprinting can create complex and patient-specific designs from a wide range of materials. The methods described herein can generate biomimetic, thin membranous structures, such as the periosteum, cornea, and epidermis with resolutions of less than one hundred fifty microns (150 μm).

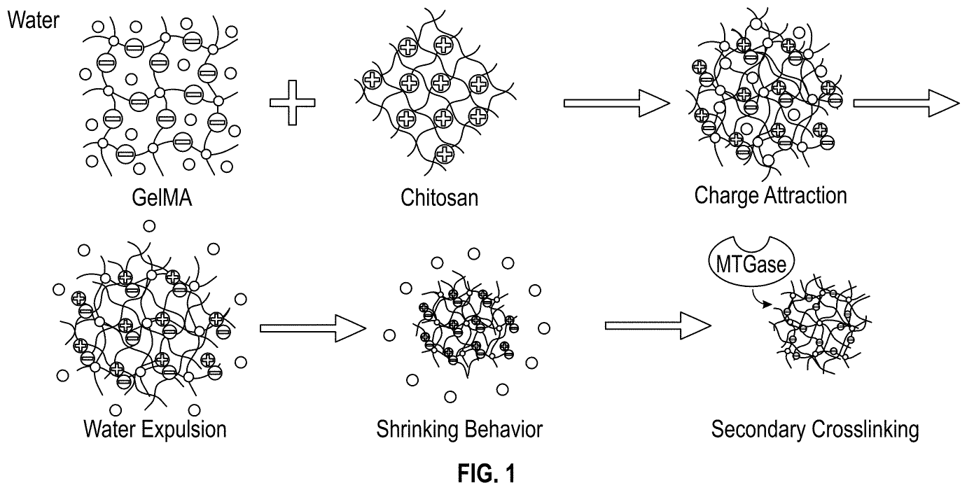

Specifically, through a mechanism similar to complex coacervation, the charges between negatively and positively charged hydrogels attract. This causes slight water expulsion from the hydrogel, and subsequent matrix shrinking, which is demonstrated in FIG. 1.

Bioprinting resolution can be enhanced by harnessing charge interactions between polyanionic and polycationic polymers. For example, gelatin methacrylate (GelMA) hydrogels are placed in chitosan solutions to induce a shrinking behavior by a complex coacervation-like mechanism. The modified structure will then undergo secondary crosslinking via microbial transglutaminase (MTGase) to maintain the enhanced resolution.

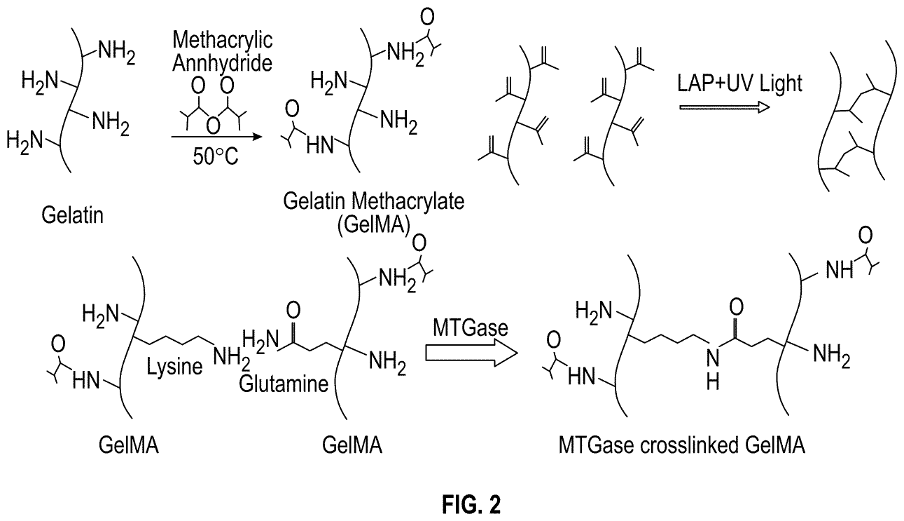

Gelatin methacrylate synthesis, primary and secondary crosslinking mechanisms are shown in the schematic of FIG. 2. Type A gelatin and methacrylic anhydride were used to synthesize GelMA, as shown in the top row of FIG. 2). While type A gelatin is positively charged at neutral pH, the addition of anionic methacrylate groups results in reduced net charge. The GelMA structure is shown below:

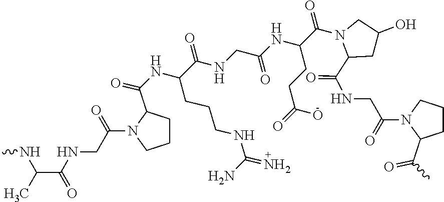

Chitosan includes a net positive charge. The chitosan structure is shown below:

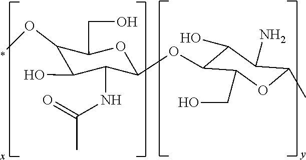

Incubation of anionic GelMA (−) in cationic chitosan (+) results in charge attraction and subsequent hydrogel shrinking. MTGase secondary crosslinking further reduces sample volume.

MTGase enzymatically crosslinks gelatin by catalyzing bond formation between lysine and glutamine residues on the gelatin backbone, as shown in the bottom row of FIG. 2. The L-lysine structure and glutamine structures are shown below:

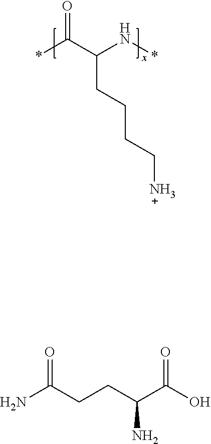

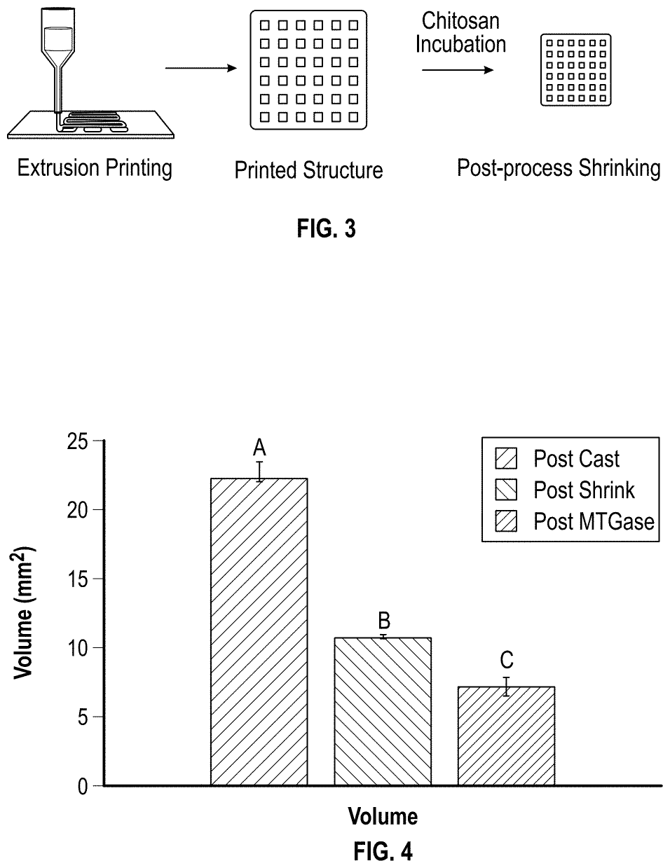

The workflow of certain aspects of this system this system consists of printing 5% GelMA hydrogels combined with 0.1% lithium phenyl-2,4,6-trimethylbenzoylphosphinate (LAP), crosslinking the structures, incubating them in 2% chitosan dissolved in 1% acetic acid for four hours, then incubating them in 5 unit/mL microbial transglutaminase overnight as shown in FIG. 3.

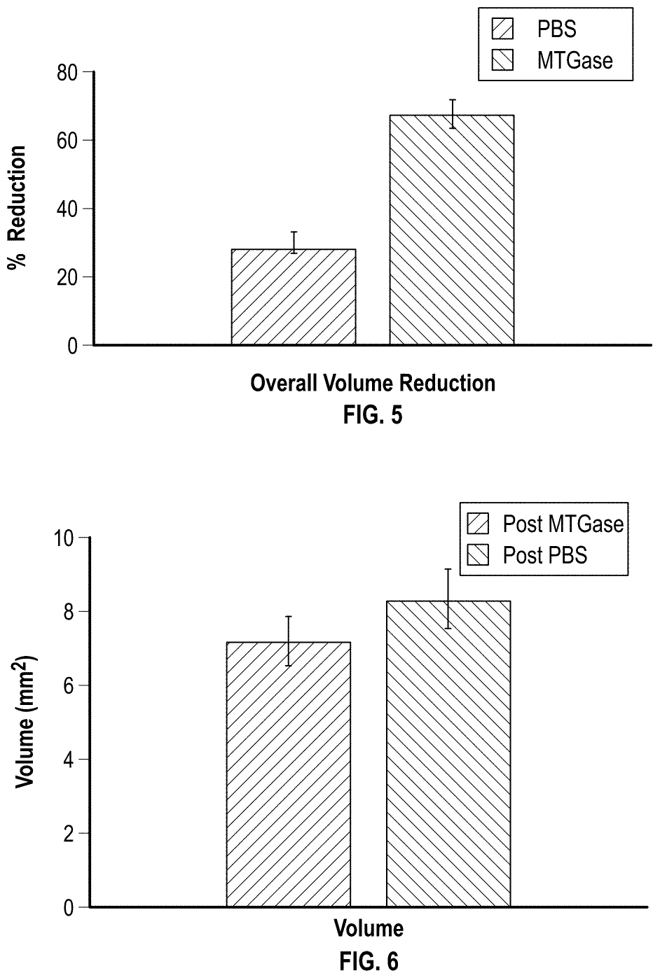

FIGS. 4-5 show the effects of shrinking and secondary crosslinking in significant volume reduction and shape maintenance.

Specifically, incubation in shrinking and secondary crosslinking solutions each result in a reduction in hydrogel sample volume, as demonstrated in FIG. 4. To determine the effect of secondary crosslinking on total sample volume reduction, samples can be incubated in the shrinking solution and compared, then placed in either MTGase or PBS. Different letters indicate statistical significance (p<0.05). Charge attraction results in significant volume reduction post shrink. Incubation in secondary crosslinking solution results in further volume reduction.

The results of FIG. 5 demonstrate that secondary crosslinking leads to a greater decrease in volume, as compared to only primary crosslinked samples. The stability of the secondary crosslinked structure was tested by incubating samples in chitosan, then MTGase, then in PBS for 24 hours. Secondary crosslinked structures have 40% greater volume reduction than singular crosslinked/shrunken gels.

Samples can be incubated in MTGase and transferred to PBS & incubated overnight. The results in FIG. 6 demonstrate only a slight increase in sample volume.

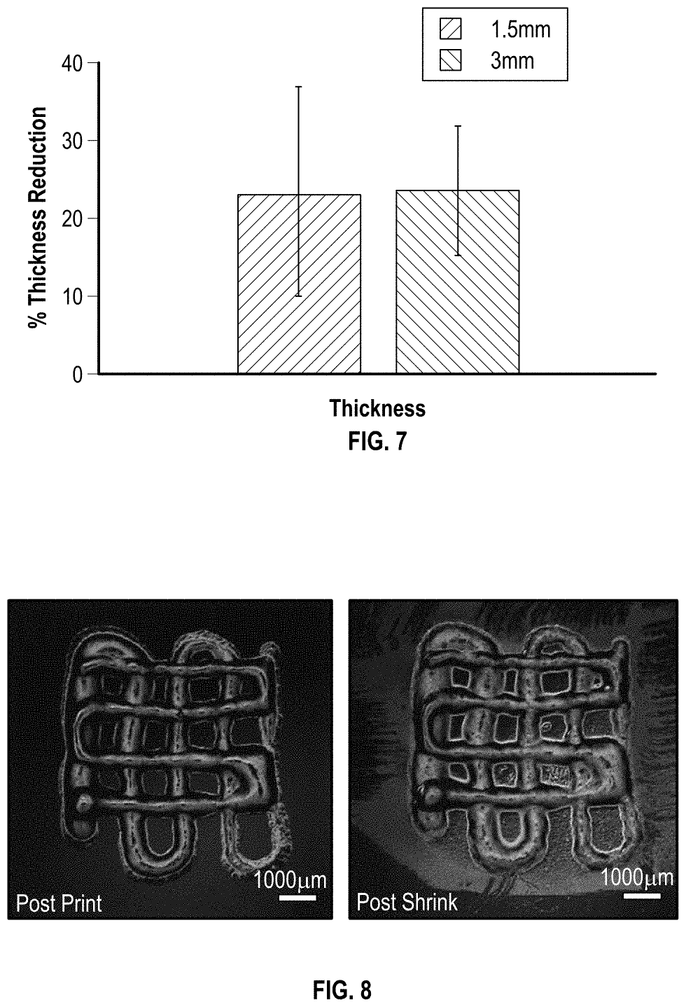

The results in FIG. 7 demonstrate that shrinking is in fact isotropic in nature. The next step was to determine how shrinking impacted printed fiber diameter.

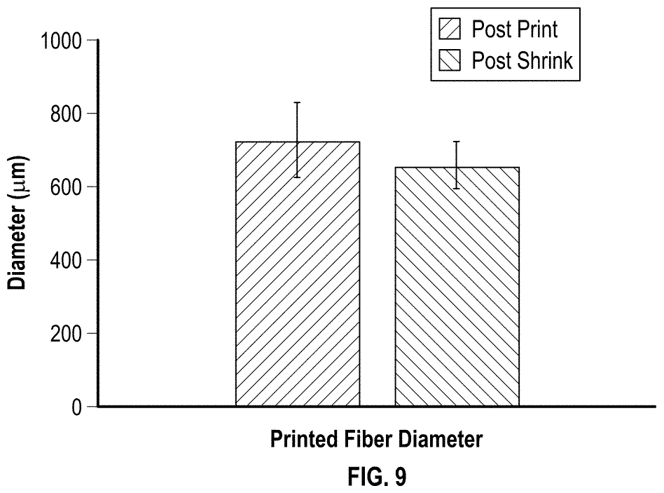

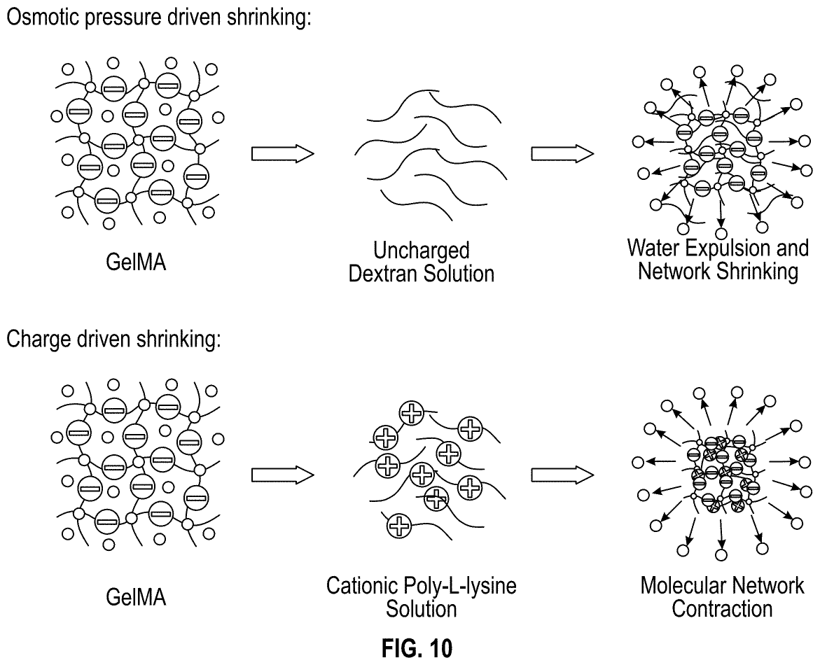

Post-printing modification results in reduction of fiber diameter. Images of printed structures are shown in FIG. 8 and the measured fiber diameter results are shown in FIG. 9. While shrinking did not lead to a significant difference in fiber diameter, this could have been due to substrate spreading on a hydrophilic surface. Future investigations will measure printed fiber diameter and height on hydrophobic surfaces to obtain a better understanding of how this process impacts printed structure properties.

It is to be appreciated that the biocompatibility of the enhancement of the resolution of extrusion-based printing of hydrogels for tissue engineering purposes allows for cellular incorporation into the shape-changing gels.

It is also to be appreciated poly-lysine can act as a potential shrinking agent that would eliminate the need for acidic chitosan as a shrinking agent. While chitosan acts as an efficient shrinking agent for GelMA-based hydrogels, its viscous and acidic nature has been shown to reduce cellular viability in the resolution enhancement of printed, cell-laden constructs. To target this issue. Poly-lysine and gelatin type A are potential alternatives. In some embodiments, poly-lysine is preferred to both chitosan and gelatin type A.

It is also to be appreciated the charge density and molecular weight of shrinking agent will impact percent reduction in sample dimensions.

It is also to be appreciated the post-processing strategies discussed herein will impact printed hydrogel fiber aspect ratio to define achievable resolution.

It is also to be appreciated certain aspects of the post-processing strategies discussed herein can be optimized through the careful selection and/or use of needle size, combined bioprinting method(s), support structure(s), and material viscosity for bioink tuning.

From the foregoing, it can be seen that the present disclosure accomplishes at least all of the stated objectives.

### EXAMPLES

GelMA was synthesized and dissolved in phosphate buffered saline (PBS) at a concentration of 5% w/v with Lithium phenyl-2,4,6-trimethylbenzoylphosphinate (LAP) added as a photoinitiator. Samples for shrinking characterization studies were cast in a cylindrical shape with a diameter of 4.3 mm and a thickness of 1.30 mm. All samples were UV crosslinked for 2 minutes after casting. Diameter and thickness were measured using calipers and volume was calculated from the measured quantities. Samples were then placed in a 2% w/v chitosan solution dissolved acetic acid to induce shrinking over 4 hours. Diameter and thickness were again measured after the shrinking period. To ensure samples would maintain the shape change, they were placed in 5U/mL microbial transglutaminase (MTGase) for secondary enzymatic crosslinking. These hydrogels were compared to a control incubation in PBS to determine the effect of secondary crosslinking on shape change behavior.

Significant decreases in sample volume were observed after shrinking and incubation in the secondary crosslinking solution. Compared to hydrogels incubated in PBS alone, the overall volume reduction of secondary crosslinked structures was 40% higher, as shown in FIG. 4. These results indicate that charge interactions between anionic and cationic hydrogels can be manipulated to create shape-changing behavior in these materials. This post-processing strategy effectively enhances the resolution of extrusion-based printing of hydrogels for tissue engineering purposes.

To determine the impact of secondary crosslinking on volume reduction, samples were incubated in PBS or MTGase after shrinking. Samples incubated in MTGase resulted in a 30% greater volume reduction than PBS samples, as shown in FIG. 5, illustrating the importance of MTGase incubation on shape maintenance of shrunken samples. The stability of the secondary crosslinked structure was tested by incubating samples in chitosan, MTGase, and then PBS. While a slight increase in sample volume was observed, MTGase incubation time can be modified to ensure maintenance of the shrunken state.

To determine whether this shrinking modification was isotropic in nature, samples of different thicknesses were casted and measured post cast and post shrink. As shown in FIG. 7, results indicate that shrinking is substantially independent of sample thickness. Casted samples indicate that shrinking behavior is isotropic.

As shown in FIG. 9, printed hydrogel measurements indicated a slight decrease in sample diameter. Hydrogels were printed on hydrophobic substrates for better shape maintenance.

### Manipulation of Hydrogel Charge Interactions for the Enhancement of Extrusion Bioprinting Resolution

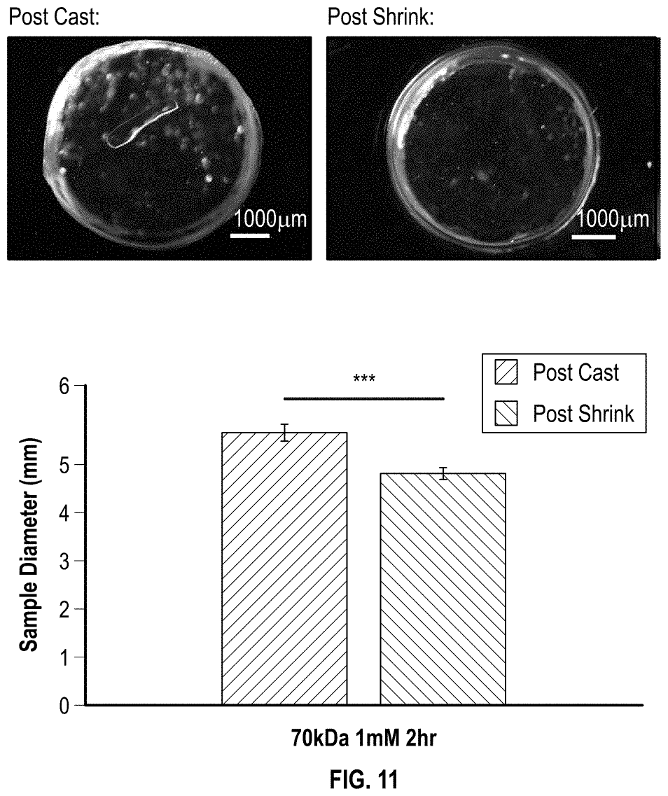

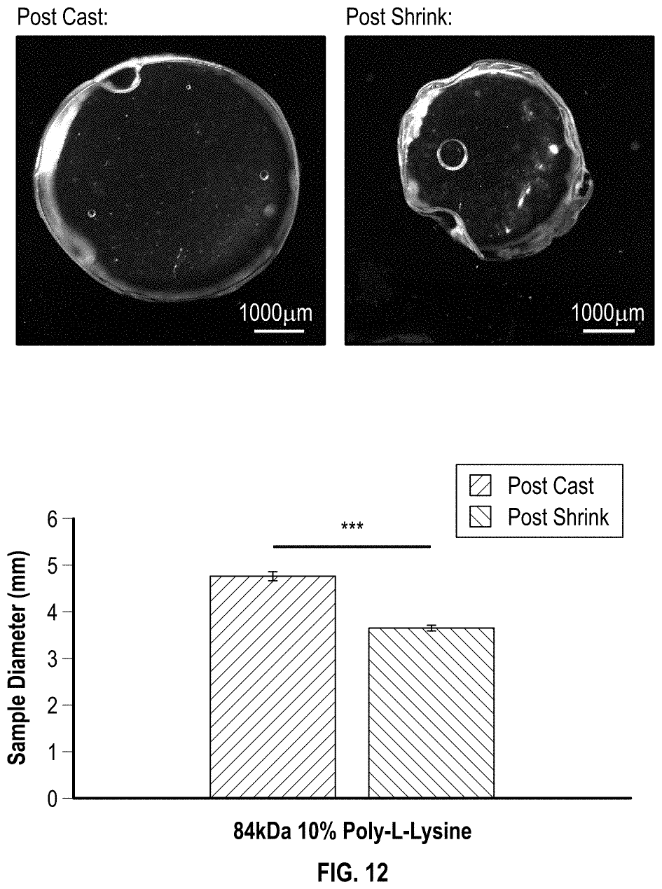

Osmotic pressure-driven shrinking only modestly shrinks GelMA network, as shown in FIG. 11. 70 kDa 1 mM uncharged shrinking results in ˜15% reduction in sample dimensions. As shown in FIG. 12, Poly-L-lysine contracts GelMA network. Poly-L-lysine exhibits the ability to reduce GelMA sample dimensions by ˜23%.

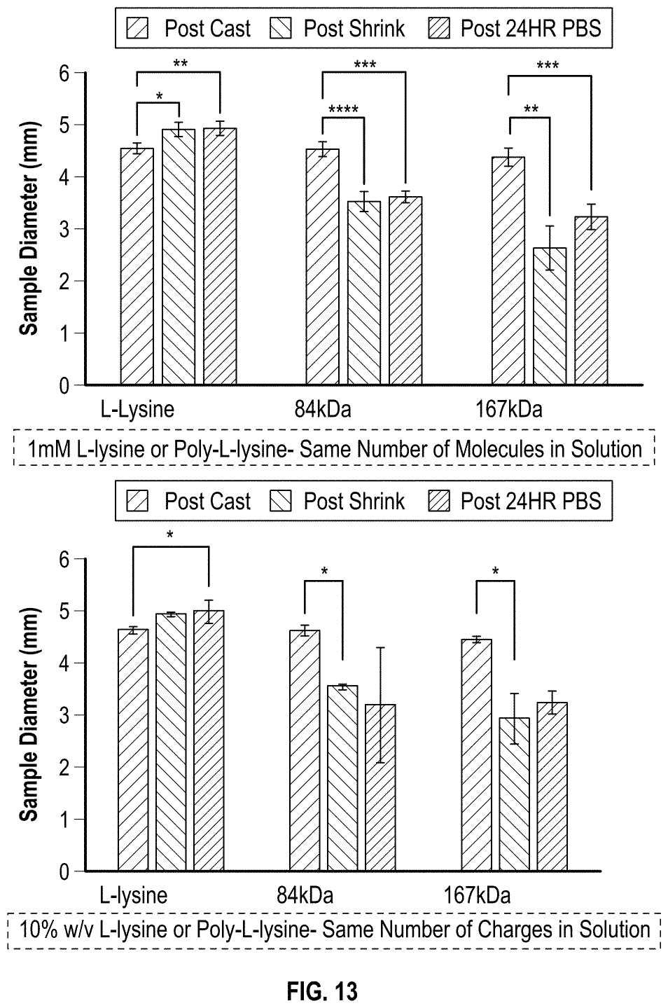

FIG. 13 demonstrates longer Poly-L-lysine molecules lead to better network collapse, regardless of weight or number of charges in solution. The top bar graph shows 1 mM L-lysine or Poly-L-lysine-same number of molecules in solution, while the bottom bar graph shows 10% w/v L-lysine or Poly-L-lysine-same number of charges in solution. 24 hour incubation in PBS was conducted to determine stability of shrunken state without secondary crosslinking after Poly-L-lysine shrinking.

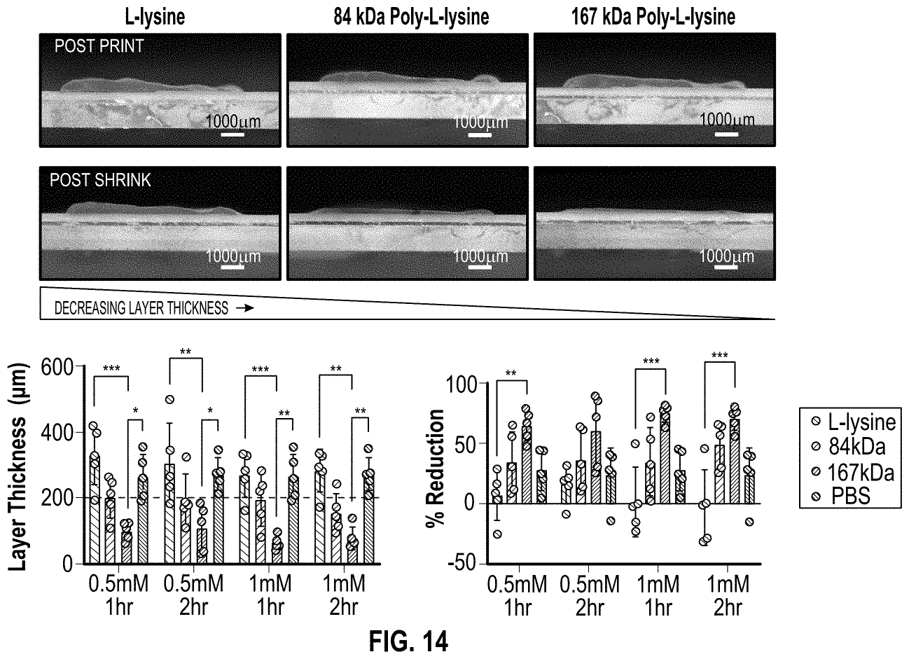

FIG. 14 captures images of Poly-L-lysine MW driving the final printed layer thickness. L-lysine has a thicker layer than 84 kDa Poly-L-lysine, which has a thicker layer than 167 Poly-L-lysine.

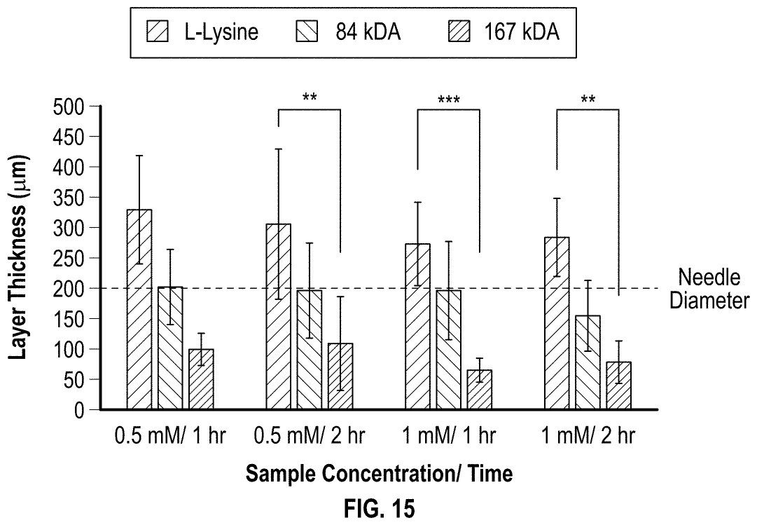

FIG. 15 demonstrates Poly-L-lysine incubation enhances extrusion print resolution. In FIG. 15, the extrusion printing resolution is between ˜150-200 μm. The final layer thickness post shrink ˜66 μm. The increased contact area of poly-L-lysine to print layer-increased shrinking. Poly-L-lysine incubation time and concentration show no significant impact, while Poly-L-lysine molecular weight drives shrinking capabilities.

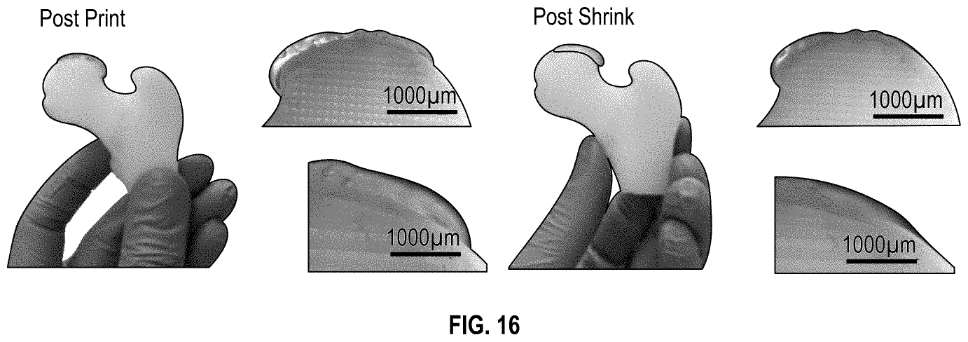

FIG. 16 captures extrusion-based printing can generate macroscale and microscale structures in the same construct.

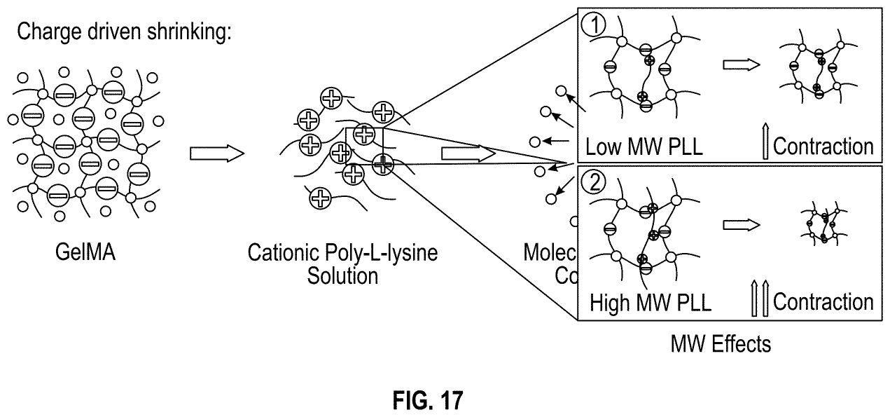

As shown in FIG. 17, with increasing MW, poly-L-lysine exhibits increased molecular network contraction on GelMA. Longer Poly-L-lysine chains allow for increased GelMA network collapse.

### Enhancing Extrusion Bioprinting Resolution by Manipulation of Hydrogel Charge Interactions

Both gelatin type A and poly-lysine were evaluated as potential replacements for chitosan as shrinking agents. Samples that were incubated in gelatin type A presented inconsistent and moderate shrinking. However, samples incubated in both 2% and 5% poly-lysine presented significant reduction in sample diameter. While samples incubated in 10% poly-lysine showed high variability in amount of shrinking, the percent reduction achieved was similar to that of chitosan. Printed fiber dimensions showed a ˜50% reduction in fiber height and fiber width.

Type B gelatin and methacrylic anhydride were used to synthesize GelMA. 5 w/v % GelMA solutions were prepared with 0.1% LAP photoinitiator and PBS. For sample dimension analysis, samples were casted in 4.5 mm diameter 1.5 mm height cylinders, crosslinked for 2 minutes, and imaged post cast. Samples were then incubated in dextran, L-lysine, or PLL solutions and imaged again post shrink.

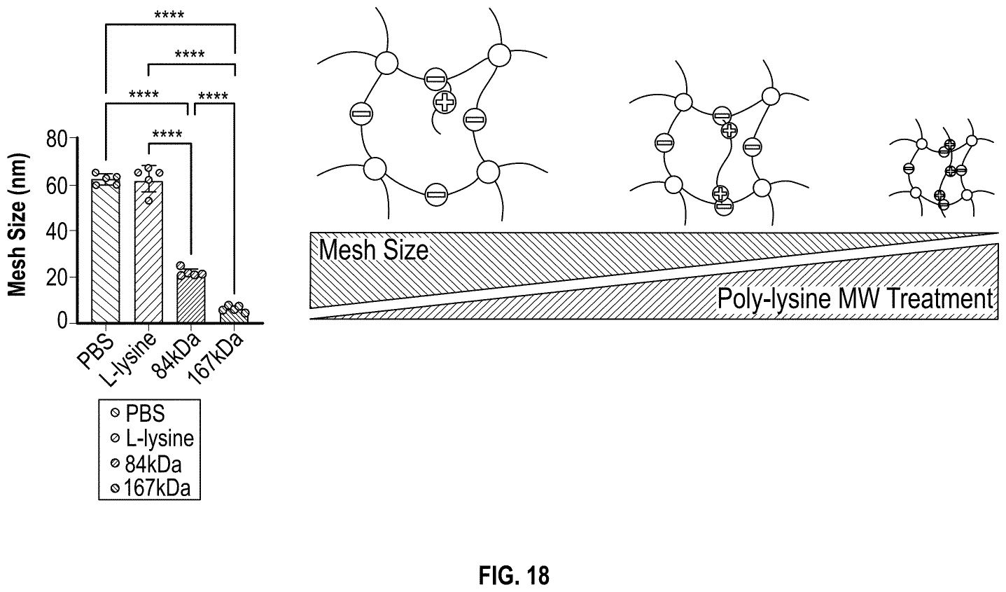

Samples of 10 mm diameter and 5 mm height were casted using PDMS molds and weighed immediately after 2 minutes of crosslinking, as shown in FIG. 18. They were then incubated in PBS for 48 hours to allow for equilibrium swelling and were weighed again. Samples were then subjected to shrinking treatment, weighed again, then lyophilized for 24 hours. Lyophilized weights were also recorded. Mesh size was calculated using the equations:

### Flory Characteristic Ratio:

$${C}_{n} = 0.95 \frac{2 {l}_{p}}{l}$$

### Molecular Weight Between Crosslinks:

$$\frac{1}{{M}_{c}} = \frac{1}{{M}_{n}} - \frac{\frac{\overline{v}}{{V}_{1}} [ ln ( 1 - {v}_{2 s} ) + {v}_{2 s} + {X}_{1} {v}_{2 s}^{2} ]}{{v}_{2 r} [ {( \frac{{v}_{2 s}}{{v}_{2 r}} )}^{\frac{1}{3}} - \frac{1}{2} ( \frac{{v}_{2 s}}{{v}_{2 r}} ) ]}$$

### Mesh Size:

$$ξ = {v}_{2 s}^{- \frac{1}{3}} * l * {( ( 2 \frac{{M}_{c}}{{M}_{r}} ) {C}_{n} )}^{\frac{1}{2}}$$

### Relaxed Mass Swelling Ratio:

$${Q}_{mr} = \frac{( {m}_{post cast} - {m}_{l y p h o l i z e d} )}{{m}_{l y p h o l i z e d}}$$

### Relaxed Volumetric Swelling:

$${Q}_{vr} = 1 + \frac{{ρ}_{p}}{{ρ}_{s}} ( {Q}_{mr} - 1 )$$

### Relaxed Polymer Volume Fraction:

$${v}_{2 r} = \frac{1}{{Q}_{vr}}$$

### Equilibrium Mass Swelling Ratio:

$${Q}_{m} = \frac{( {m}_{peq swell / shrink} - {m}_{lypholized} )}{{m}_{lypholized}}$$

### Equilibrium Volumetric Swelling:

$${Q}_{v} = 1 + \frac{{ρ}_{p}}{{ρ}_{s}} ( {Q}_{m} - 1 )$$

### Equilibrium Polymer Volume Fraction:

$${v}_{2 s} = \frac{1}{{Q}_{v}} \\ {ρ}_{s} = 1.01 g / {cm}^{3} {ρ}_{p} = 1.35 g / {cm}^{3} \overline{v} = 0.7407 mL / {gV}_{1} = 18.01 mL / mol {X}_{1} = 0.497 {M}_{n} = 63565 g / mol l = 4.28 \dot{A} {C}_{n} = 8.8785 {M}_{r} = 91.9 g / mol {l}_{p} = 20 \dot{A} \\ wherein , , , , {ρ}_{s} = 1.01 g / {cm}^{3} {ρ}_{p} = 1.35 g / {cm}^{3} \overline{v} = 0.7407 mL / {g V}_{1} = 18.01 mL / mol {X}_{1} = 0.49 7 {M}_{n} = 63565 g / mol l = 4.28 \dot{A} {C}_{n} = 8.8785 {M}_{r} = 91.9 g / mol {l}_{p} = 20 \dot{A} , , , , ,$$

and

5% GelMA with 0.1% LAP solutions were prepared and placed in an extrusion printer barrel. Barrels were precooled to 10° C. for 1 hr. Samples were printed on Teflon coverslips to reduce sample-substrate interactions. Samples were printed at 10° C., 6-8 psi, 4-5 mm/s speed with the Advanced Solutions BioAssembly Bot in single layer structures. Samples were imaged post print and shrink.

Samples were conducted with n=5 with 8-10 technical measurements. Statistical analyses were conducted in GraphPad Prism. Samples were assessed for normality using Shapiro-Wilk test. Nonnormal data sets were evaluated with Wilcoxon Rank and Kruskal Wallis tests. Normal data sets were evaluated with t-tests and ANOVAs.

Data from casting experiments suggests that charge-driven shrinking heavily relies on the molecular weight of the shrinking agent (PLL). When comparing trends between weight percent solutions and molar solutions, longer PLL chains exhibit greater ability to contract the GelMA network, regardless of number of charges in solution. To further explore this phenomenon, mesh size of hydrogels treated with different PLL molecular weights were investigated. Resulting data demonstrate smaller mesh size with increased molecular weight treatment.

While osmotic pressure-driven shrinking demonstrates a modest reduction in sample dimensions, charge driven shrinking with PLL demonstrated significant differences in sample dimension post cast to post shrink. When investigating the impact of PLL molecular weight on shrinking, the same trend in samples treated with the same number of charges in solution (w/v %) as samples treated with the same number of molecules in solution (mM) were observed. Longer PLL molecules have greater ability to contract the GelMA network, as evidenced in the mesh size analysis.

Factorial design analysis (the results of which are seen at the bottom of FIG. 14) of printed structures demonstrated that the only significant factor in printed shrinking was molecular weight, regardless of concentration or incubation time. This further suggests that longer PLL molecules better contract GelMA hydrogel networks.

### Applications

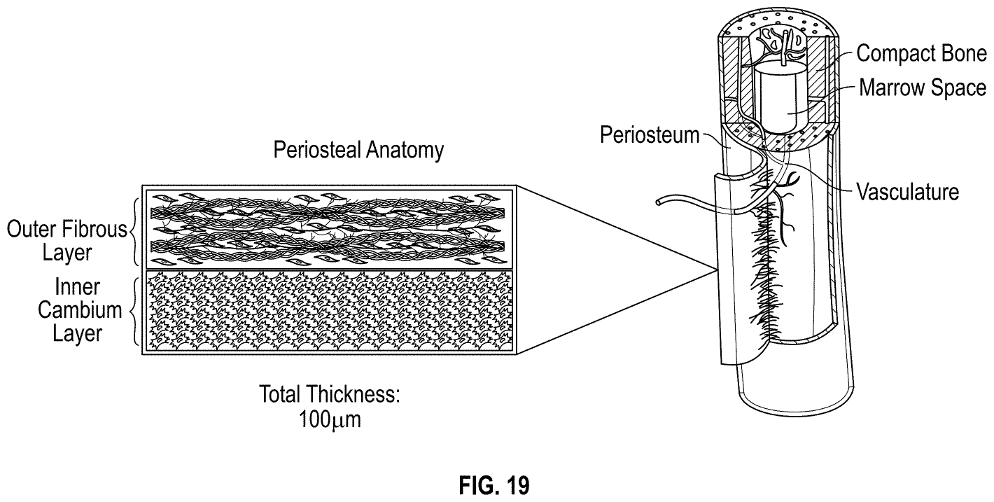

FIG. 19 in particular shows an example schematic of a tissue engineering periosteum. The periosteal anatomy comprises an outer fibrous layer and an inner cambium layer. The periosteum, the outer layer surrounding bone tissue, is about one hundred microns (100 μm) in total thickness. This structure contains both an osteoprogenitor cell-rich inner layer, and a fibrous outer layer consisting of collagen, fibroblasts, and vasculature.

### Glossary

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

Biocompatibility, as used herein, is a general term describing the property of a material being compatible with living tissue. For example, materials that are biocompatible with humans do not produce a toxic or immunological response when exposed to the body or bodily fluids.

Gelatin methacrylate (GelMA) is a photopolymerizable hydrogel comprised of modified natural ECM components, making it a potentially attractive material for tissue engineering applications.

Chitosan is a linear polysaccharide composed of randomly distributed β-(1→4)-linked D-glucosamine (deacetylated unit) and N-acetyl-D-glucosamine (acetylated unit).

Lysine is an α-amino acid that is a precursor to many proteins. It contains an α-amino group (which is in the protonated —NH₃⁺ form under biological conditions), an α-carboxylic acid group (which is in the deprotonated —COO⁻ form under biological conditions), and a side chain lysyl ((CH₂)₄NH₂), classifying it as a basic, charged (at physiological pH), aliphatic amino acid. It is encoded by the codons AAA and AAG. Like almost all other amino acids, the α-carbon is chiral. Generally, lysine may refer to either enantiomer or a racemic mixture of both. For purposes of the present disclosure, it is to be appreciated lysine can refer to the biologically active enantiomer L-lysine, where the α-carbon is in the S configuration. The human body cannot synthesize lysine.

Microbial transglutaminase (mTGase) is an enzyme that catalyzes site-specific protein derivatization at specific glutamines.

Lithium phenyl-2,4,6-trimethylbenzoylphosphinate (LAP) is a water soluble, cytocompatible, Type I photoinitiator for use in the polymerization of hydrogels or other polymeric materials.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. A method of generating a shrinking response comprising:
allowing for cells to be incorporated into shape-changing gels;
manipulating charge interactions between the shape-changing gels to induce a shrinking behavior and to expel water from the shape-changing hydrogels; and
generating a three-dimensional construct using an extrusion-based bioprinting process.

2. The method of claim 1 further comprising incubating shrunken samples with a secondary crosslinking mechanism to provide a permanent shape to said shape-changing gels.

3. The method of claim 2 wherein the secondary crosslinking mechanism is an enzymatic crosslinking mechanism.

4. The method of claim 3 wherein the enzymatic crosslinking mechanism comprises microbial transglutaminase (MTGase).

5. The method of claim 1 wherein the shape-changing gels comprise a polyanionic hydrogel and a polycationic hydrogel.

6. The method of claim 5 wherein the polyanionic hydrogel comprises gelatin methacrylate (GelMA).

7. The method of claim 5 wherein the polycationic hydrogel comprises chitosan or poly-L-lysine.

8. The method of claim 1 further comprising a primary crosslinking mechanism reacts to UV exposure.

9. The method of claim 8 wherein the primary crosslinking mechanism comprises Lithium phenyl-2,4,6-trimethylbenzoylphosphinate (LAP).

10. The method of claim 1 wherein the three-dimensional construct is a membranous tissue selected from the group consisting of: periosteum, cornea, and epidermis.

11. The method of claim 10 further comprising enhancing the resolution of bioprinted hydrogels in the extrusion-based printing such that the membranous tissue is less than one hundred microns (100 μm) in thickness.

12. The method of claim 1 wherein three-dimensional construct comprises a bio-ink.

13. A three-dimensional construct comprising:
a polyanionic hydrogel and a polycationic hydrogel;
cells that can be incorporated into the polyanionic hydrogel; and
a permanent shape formed from primary UV crosslinking and secondary enzymatic crosslinking.

14. The three-dimensional construct of claim 13 wherein the polyanionic hydrogel is anionic gelatin methacrylate (GelMA) and the polycationic hydrogel cationic comprises poly-L-lysine (PLL).

15. The three-dimensional construct of claim 13 wherein longer PLL molecules are employed to better contract GelMA hydrogel networks.

16. A method of demonstrating a shrinking response in a three-dimensional bioprinted material comprising:
synthesizing gelatin methacrylate (GelMA) from type B gelatin and methacrylic: anhydride, dialyzed, and lyophilized;
dissolving the GOMA in a solution with a photoinitiator;
casting samples;
after casting said samples, UV crosslinking the samples by activating the photoinitiator;
immersing said crosslinked samples in a cationic chitosan solution, gelatin type A, or poly-lysine.

17. The method of claim 16 wherein the solution comprises gelatin methacrylate dissolved in phosphate buffered saline (PBS) at a concentration of 5% w/v with lithium phenyl-2,4,6-trimethylbenzoylphosphinate (LAP) at a concentration of 0.1% w/v.

18. The method of claim 16 wherein the samples are cast in a cylindrical shape with a diameter equal to or less than 4.5 mm/s and a thickness equal to or less than 1.50 mm.

19. The method of claim 16 further comprising printing at a temperature of approximately 10° C., speed of approximately 4-5 mm/s, and a pressure of approximately 6-8 psi in the shape of individual fibers.

20. The method of claim 16 wherein the immersing occurs between 1-4 hours of incubation time.
